(19) United States
(12) Patent Application Publication
(10) Pub. No.: US 2018/0214412 A1
(43) Pub. Date: Aug. 2, 2018

(54) IMMEDIATE RELEASE CANNABINOID FORMULATIONS

(71) Applicant: CannTab Therapeutics Limited, Toronto (CA)

(72) Inventors: Jeff Renwick, Thornhill (CA); Robert Scott Lefler, Brantford (CA)

(21) Appl. No.: 15/878,396

(22) Filed: Jan. 23, 2018

Related U.S. Application Data
(60) Provisional application No. 62/449,395, filed on Jan. 23, 2017.

(51) Int. Cl. A61K 31/353; A61K 9/20; A61K 31/05

(52) U.S. Cl. CPC A61K 31/353; A61K 9/2059; A61K 9/2009; A61K 31/05; A61K 9/2054; A61K 9/2027; A61K 9/2031; A61K 9/2018

(57) ABSTRACT

The present invention relates to immediate release pharmaceutical compositions comprising one or more natural or synthetic cannabinoids, and one or more pharmaceutically acceptable excipients. More specifically, the invention relates to immediate release pharmaceutical compositions comprising cannabinoids and a process for preparation thereof.

### FIELD OF THE INVENTION

The present invention relates to immediate release pharmaceutical compositions comprising a cannabinoid, one or more release modifying agents and one or more pharmaceutically acceptable excipients. More specifically, the invention relates to immediate release pharmaceutical compositions comprising cannabidiol, tetrahydrocannabinol or tetrahydrocannabinovarin and a process for preparation thereof.

### BACKGROUND OF THE INVENTION

Cannabinoids are a class of diverse chemical compounds that act on cannabinoid receptors on cells that repress neurotransmitter release in the brain. The most notable cannabinoid found in Cannabis is the phytocannabinoid, tetrahydrocannabinol (THC), the primary psychoactive compound of cannabis. Cannabidiol (CBD) is another major constituent of the plant. There are at least 85 different cannabinoids isolated from Cannabis, exhibiting varied effects. From Wikipedia http://en.wikipedia.org/wiki/Tetrahydrocannabinol accessed May 25, 2015.

As used herein, “Cannabis” includes wild type Cannabis sativa and variants thereof, including cannabis chemotypes which naturally contain widely different amounts of the individual cannabinoids.

The term “cannabinoids” as used herein includes but is not limited to purified, pharmaceutical grade substances, which may be obtained by purification from a natural source or via synthetic means. The formulations according to the invention may be used for delivery of extracts of cannabis plants and also individual cannabinoids, or synthetic analogues thereof, whether or not derived from cannabis plants, and also combinations of cannabinoids.

Delta-9-Tetrahydrocannabinol (one isomer of this compound is known as dronabinol) is a naturally occurring compound and is the primary active ingredient in marijuana. Marijuana is dried hemp plant Cannabis Sativa. The leaves and stems of the plant contain cannabinoid compounds (including dronabinol). Dronabinol has been approved by the Food and Drug Administration for the control of nausea and vomiting associated with chemotherapy and for appetite stimulation of patients suffering from wasting syndrome. Synthetic dronabinol is a recognized pharmaceutically active ingredient, but natural botanical sources of cannabis rather than synthetic THC are also known in the art.

Dronabinol is a light yellow resinous oil that is sticky at room temperature and hardens upon refrigeration. Dronabinol is insoluble in water and is usually formulated in sesame oil. It has a pKa of 10.6 and an octanol-water partition coefficient: 6,000:1 at pH 7. After oral administration, dronabinol has an onset of action of approximately 0.5 to 1 hours and peak effect at 2 to 4 hours. Duration of action for psychoactive effects is 4 to 6 hours, but the appetite stimulant effect of dronabinol may continue for 24 hours or longer after administration.

Dronabinol is the international nonproprietary name for a pure isomer of THC, (−)-trans-Δ9-tetrahydrocannabinol, which is the main isomer found in cannabis. Synthesized dronabinol is marketed as Marinol (a registered trademark of Solvay Pharmaceuticals).

U.S. Pat. No. 9,029,423 disclose numerous therapeutic uses and effects of cannabinoids, including, at least, modulating or impacting: CB1 (Brain receptors), CB2 (Peripheral receptors), CNS Effects, Anticonvulsant, Antimetrazol, Anti-electroshock, Muscle Relaxant, Antinociceptive, Catalepsy, Psychoactive, Antipsychotic, Neuroprotective antioxidant activity, Antiemetic, Sedation (reduced spontaneous activity), Appetite stimulation, Appetite suppression, Anxiolytic, Cardiovascular Effects, Bradycardia, Tachycardia, Hypertension, Hypotension, Anti-inflammatory, Immunomodulatory/anti-inflammatory activity, Cox 1, Cox 2, TNFα Antagonism, and Glaucoma.

U.S. Pat. No. 6,403,126 (incorporated herein by reference in its entirety) discloses methods of extracting and purifying cannabinoids from Cannabis using organic solvents including: a petroleum derived hydrocarbon; toluene; trimethylpentane; low molecular weight alcohol; ethanol; low molecular weight chlorinated hydrocarbon; and dichloromethane.

U.S. Pat. Application No. 20120231083 discloses a sustained release medicament which results in delivery of a therapeutic level of one or more cannabinoids during a clinically relevant therapeutic window.

U.S. Pat. Application No. 20060257463 discloses a method of transmucosally delivering a cannabinoid to a subject in need of such treatment.

Pharmaceutical compositions comprising the active cannabinoid pharmaceutical ingredient, crystalline trans-(±)-Δ9-tetrahydrocannabinol, and formulations thereof are disclosed in WO 2006133941. In specific embodiments, the crystalline trans-(±)-Δ9-tetrahydrocannabinol administered according to the methods for treating or preventing a condition such as pain can have a purity of at least about 98% based on the total weight of cannabinoids.

U.S. Pat. Application No. 20140100269 discloses oral cannabinoid formulations, including an aqueous-based oral dronabinol solution, that are alleged to be stable at room or refrigerated temperatures and may possess improved in vivo absorption profiles with faster onset and lower inter-subject variability.

U.S. Pat. No. 8,632,825 discloses the use of a combination of cannabinoids, particularly tetrahydrocannabinol (THC) and cannabidiol (CBD), in the manufacture of a medicament for use in the treatment of cancer. In particular the cancer to be treated is a brain tumor, more particularly a glioma, more particularly still a glioblastoma multiforme.

U.S. Pat. No. 6,630,507 discloses that cannabinoids have antioxidant properties. This property makes cannabinoids useful in the treatment and prophylaxis of wide variety of oxidation associated diseases, such as ischemic, age-related, inflammatory and autoimmune diseases. The cannabinoids are disclosed to have particular application as neuroprotectants, for example in limiting neurological damage following ischemic insults, such as stroke and trauma, or in the treatment of neurodegenerative diseases, such as Alzheimer's disease, Parkinson's disease and HIV dementia. Non-psychoactive cannabinoids, such as cannabidoil, are disclosed as particularly advantageous to use because they avoid toxicity that is encountered with psychoactive cannabinoids at the high doses useful in the method of the disclosure.

U.S. Pat. No. 8,808,734 discloses stable, fast-acting liposomal and micelle formulations of cannabinoids or cannabinoid analogues.

U.S. Pat. No. 6,747,058 discloses stable composition for inhalation therapy comprising delta-9-tetrahydrocannabinol and semi-aqueous solvents therefor.

U.S. Pat. No. 6,946,150 and related patents and applications, all of which are incorporated herein by reference, disclose various sprays and other related dosage forms of various cannabinoids. See U.S. Pat. No. 7,709,536, U.S. Pat. No. 8,211,946, U.S. Pat. No. 8,603,515, US20040034108, US20060068034, US20100196488, US20130109747, US20130245109, US20140296351, and US20160068321.

DOSAGE AND ADMINISTRATION OF DRONABINOL FROM FDA DOCUMENT NDA 18-651/S-021; 500012 Rev September 2004:
Appetite Stimulation: Initially, 2.5 mg Dronabinol Capsules should be administered orally twice daily (b.i.d.), before lunch and supper. For patients unable to tolerate this 5 mg/day dosage, the dosage can be reduced to 2.5 mg/day, administered as a single dose in the evening or at bedtime. If clinically indicated and in the absence of significant adverse effects, the dosage may be gradually increased to a maximum of 20 mg/day, administered in divided oral doses. Caution should be exercised in escalating the dosage because of the increased frequency of dose-related adverse experiences at higher dosages.
Antiemetic: Best administered at an initial dose of 5 mg/m2, given 1 to 3 hours prior to the administration of chemotherapy, then every 2 to 4 hours after chemotherapy is given, for a total of 4 to 6 doses/day. Should the 5 mg/m2 dose prove to be ineffective, and in the absence of significant side effects, the dose may be escalated by 2.5 mg/m2 increments to a maximum of 15 mg/m2 per dose. Caution should be exercised in dose escalation, however, as the incidence of disturbing psychiatric symptoms increases significantly at maximum dose.

Despite all of the work on cannabinoids and dronabinol, there is a need in the art for solid, monolithic dosage forms that have an improved absorption profile with faster onset, improved release profiles and lower inter-subject variability than currently available gelatin capsules.

Accordingly, there exists a need in the art to provide a monolithic, matrix- based immediate release oral dosage form that provides for improved bioavailability of cannabidiol suitable for oral administration.

### SUMMARY OF THE INVENTION

The present invention, is directed to a immediate release oral monolithic starch acetate containing tablets comprising a therapeutically effective amount of cannabidiol, and excipients.

The present invention, is further directed to an immediate-release drug composition comprising a monolithic matrix tablet comprising cannabidiol containing granules and extragranular excipients wherein the granules comprise:
cannabidiol;
dibasic calcium phosphate;
lactose monohydrate;
pregelatinized modified starch; and
hydroxyl ethyl cellulose; and where the extragranular excipients comprise about:
carbomer homopolymer;
polyethylene oxide;
hypromellose; and
methacrylic acid copolymer.

### DETAILED DESCRIPTION

The present invention comprises one or more cannabinoids in an immediate-release monolithic tablet dosage form.

Preferably the one or more cannabinoids are present in the form of at least one extract from at least one cannabis plant.

The dosage form may also contain, in addition to the cannabinoid(s), a further active agent, which may be an opiate, for example morphine.

In one aspect of the present invention, one or more phytocannabinoids are selected from the group consisting of tetrahydrocannabivarin (THCV) and cannabidiol (CBD).

A preferred daily dose of THCV is at least 1.5 mg, more preferably at least 5 mg through 10 mg to 15 mg or more.

In another aspect, the THCV maybe used in combination with at least a second, therapeutically effective cannabinoid, preferably CBD.

The CBD may be present in an amount which will provide a daily dose of 200 mg, more preferably 600 mg and as much as 800 mg or even more.

The cannabinoids may be present as pure or isolated cannabinoids or in the form of plant extracts. Where a plant extract is used it is preferable that the THC content is less than 5% by weight of the total cannabinoids, more preferably less than 4% through 3%, 2% and 1%. THC can be selectively removed from extracts using techniques such as chromatography.

Matrix-based formulation systems may incorporate monolithic matrix systems or coating systems. Monolithic matrix systems include a polymer matrix containing dispersed or dissolved drug. Either hydrophilic or insoluble matrix systems may be used. Coated systems include a drug-containing core enclosed within a polymer barrier coat. These coating systems can be simple diffusion/erosion systems or osmotic systems where the drug core is contained within a semipermeable polymer membrane with a mechanical/laser drilled hole for drug release, driven by osmotic pressure generated within the tablet core.

Drug embedded matrix tablets are one of the least complicated approaches for obtaining high bioavailability dosage forms and are widely used and preferred when achievable. Polymers and release retarding materials used as matrix formers in matrix tablets play a vital role in controlling the drug release from the tablets. Though a variety of polymeric materials are available to serve as release controlling matrix materials, there is a continued need to develop new, safe and effective release retarding matrix dosage forms for preparing simple monolithic matrix tablets for improved release and bioavailability. The immediate release formulations of the present invention represent a significant improvement over existing cannabinoid formulations.

Granule Ingredients

Cannabinoids

Preferred cannabinoids include, but are not limited to, tetrahydrocannabinoids, their precursors, alkyl (particularly propyl) analogues, cannabidiols, their precursors, alkyl (particularly propyl) analogues, and cannabinol. In a preferred embodiment the formulations may comprise any cannabinoids selected from tetrahydrocannabinol, Δ9-tetrahydrocannabinol (THC), Δ8-tetrahydrocannabinol, Δ9-tetrahydrocannabinol propyl analogue (THCV), cannabidiol (CBD), cannabidiol propyl analogue (CBDV), cannabinol (CBN), cannabichromene, cannabichromene propyl analogue and cannabigerol, or any combination of two or more of these cannabinoids.

More preferably the formulations may contain THC and/or THCV and/or CBD.

In a preferred embodiment the formulations may contain specific, pre-defined ratios by weight of different cannbinoids, e.g. specific ratios of CBD to THC, or tetrahydrocannabinovarin (THCV) to cannabidivarin (CBDV), or THCV to THC. Certain specific ratios of cannabinoids have been found to be clinically useful in the treatment or management of specific diseases or medical conditions.

It has particularly been observed by the present applicant that combinations of specific cannabinoids are more beneficial than any one of the individual cannabinoids alone. Preferred embodiments are those formulations in which the amount of CBD is in a greater amount by weight than the amount of THC.

Certain formulations contain THC and CBD in defined ratios by weight. Preferred formulations contain the following ratios by weight of THC and CBD:—greater than or equal to 19:1 THC:CBD, greater than or equal to 19:1 CBD:THC, 4.5:1 THC:CBD, 1:4 THC:CBD and 1:2.7 THC:CBD. Other ratios may be chosen depending on the particular application.

Preferred formulations may include natural extracts of cannabis.

Starch Acetate

A key aspect of this invention is its unique combination of rate controlling polymers and modified starch which is acid digestion resistant and remains undigested in the acidic region of stomach and intestine so that it holds the drug content and improves bioavailability.

Starch acetate as a rate controlling polymer has recently been studied but the specific combination of pregelatinized starch acetate with rate controlling polymers of the present invention allow an improved immediate release drug delivery system of cannabidiol in a monolithic matrix tablet to simply and cost effectively improve on the inadequate delivery systems currently employed.

Starch is a natural polymer which possesses many unique properties. Some synthetic polymers are biodegradable and can be tailor-made easily. Therefore, by combining the individual advantages of starch and polymers, starch-based completely biodegradable polymers are useful for the present invention.

Starches are also used in the food manufacturing industry for processing, and as food thickeners or stabilizers. There are many other diverse uses for starches in the manufacturing industry.

Starch is regenerated from carbon dioxide and water by photosynthesis in plants. Owing to its complete biodegradability, low cost and renewability, starch is considered as a promising candidate for developing sustainable materials. In view of this, starch has been receiving growing attention since the 1970s. Only in the last few years has starch received attention by drug formulators.

Starch is mainly composed of two homopolymers of D-glucose: amylase, a mostly linear α- D(1, 4′)-glucan and branched amylopectin, having the same backbone structure as amylose but with many α-1, 6′-linked branch points. Starch chains have a lot of hydroxyl groups, including two secondary hydroxyl groups at C-2 and C-3 of each glucose residue, as well as one primary hydroxyl group at C-6 when it is not linked. The available hydroxyl groups react with alcohols: they can be oxidized and reduced, and may participate in the formation of hydrogen bonds, ethers and esters.

Starch has different proportions of amylose and amylopectin ranging from about 10-20% amylose and 80-90% amylopectin depending on the source. Amylose is soluble in water and forms a helical structure. Starch occurs naturally as discrete granules since the short branched amylopectin chains are able to form helical structures which crystallize. Starch granules exhibit hydrophilic properties and strong inter-molecular association via hydrogen bonding formed by the hydroxyl groups on the granule surface.

Starches are used in the pharmaceutical industry for a variety of uses, such as an excipient, a tablet and capsule diluent, a tablet and capsule disintegrant, a glidant, or as binder. Starches also absorb water rapidly, allowing tablets to disintegrate appropriately. Dave R H. Overview of pharmaceutical excipients used in tablets and capsules. Drug Topics (online). Advanstar. 10/24/2008 http://drugtopics.modernmedicine.com/drugtopics/Top+News/Overview-of-pharmaceutical-excipients-used-in-tabl/ArticleStandard/Article/detail/561047.

Modified starches have been used for various pharmaceutical purposes such as fillers, superdisintegrants and matrix formers in capsules and tablet formulations. Starch-based biodegradable polymers, in the form of microsphere or hydrogel, are suitable for drug delivery. Crosslinked starch glycolate (sodium salt) is an anionic polymer and produced by crosslinking and carboxymethylation of potato starch. In contrast to the starch of the present invention, in the state of the pharmaceutical art, the sodium starch glycolate from potato is preferred. Crosslinked starch glycolate is used in more than 155 drugs in the U.S. market including Primojel® (DMV-Fonterra) Acyclovir (Zovirax®); theophylline (Theo-Dur®); diltiazem (Cardizem® LA); cimetidine (Tagamet®); fenofibrate (Lipofen®); metoprolol tartrate (Lopressor®) mH. Omidian and K. Park in Juergen Siepmann I Ronald A. Siegel Michael J. Rathbone Editors Fundamentals and Applications of Controlled Release Drug Delivery.

Raw starch does not form a paste with cold water and therefore requires cooking if it is to be used as a food thickening agent. Pregelatinized starch, mostly from maize, has been cooked and dried. Pregelatinized starches are highly digestible. Used in instant puddings, pie fillings, soup mixes, salad dressings, sugar confectionery, and as a binder in meat products. Nutritional value is the same as that of the original starch. The result is a multipurpose excipient combining the dilution and disintegration power of native starch with new functionalities, such as flowability and controlled cohesive power. Pregelatinized starches are preferred for the present invention. David A Bender. Starch, Pregelatinized. A Dictionary of Food and Nutrition. 2005. Retrieved from Encyclopedia.com.

One of the important modifications of starch is acetylated starch. Starch acetate has excellent bond forming ability and has been used in the food industry extensively. One method of synthesizing starch acetate is to mix and reflux for 5 h at 150° C. plant starch, excess acetic anhydride and sodium hydroxide 50% solution. The reaction mixture is added to cold water to precipitate the starch acetate formed. The product is collected by vacuum filtration, washed repeatedly with water and dried at 80° C. for 2 h. Starch acetate may be characterized by determining the extent of acetylation and degree of substitution and by IR spectra. Solubility characteristics may also be tested.

Recently, starch acetate was synthesized, characterized and evaluated as rate controlling matrix former for controlled release nifedipine. Matrix tablets of nifedipine were formulated employing starch acetate in different proportions of drug and polymer and the tablets were evaluated for drug release kinetics and mechanism. Nifedipine is an effective and widely prescribed antianginal drug that requires controlled release owing to its short biological half life of 2.5 h. A few sustained release formulations of nifedipine are available commercially. Starch acetate was found suitable as matrix former for controlled release and the matrix tablets of nifedipine formulated employing starch acetate gave controlled release of nifedipine over 24 h and fulfilled the official release specification of nifedipine extended release tablets. Synthesis, Characterization And Evaluation Of Starch Acetate As Rate Controlling Matrix Former For Controlled Release Of Nifedipine Chowdary and Radha Int. J. Chem. Sci.: 9(2), 2011, 449-456.

Starch acetate is insoluble in water, aqueous buffers of pH 1.2 and 7.4, methanol, petroleum ether, dichloromethane and cyclohexane. It is freely soluble in chloroform.

Starch acetates have mostly been investigated as film-forming coatings using starch acetates (DS 2.8) in combination with commonly used plasticizers on the physical properties of starch acetate films have been evaluated. Starch acetate films are tougher and stronger than ethylcellulose films at the same plasticizer concentration. Also, in most cases, the water vapor permeability of starch acetate films was lower than that of ethylcellulose films. Due to the good mechanical properties, low water vapor, and drug permeabilities of the films, starch acetate seems to be a promising film-former for pharmaceutical coatings. The toughness of the films may result from their dense film structure, which is due to strong interaction forces between adjacent SA molecular chains. J Pharm Sci. 2002 Jan;91(1):282-9. Starch acetate—a novel film-forming polymer for pharmaceutical coatings. Tarvainen M, Sutinen R, Peltonen S, Tiihonen P, Paronen P.

Deformation during powder volume reduction, strain-rate sensitivity, intrinsic elasticity of the materials, and tensile strength of the tablets have been examined with the use of starch acetate powders as tablet excipients. Starch acetate with the lowest degree of substitution (ds) still possessed characteristics of native starch granules. The properties of more highly substituted starch acetates depend on precipitation and drying processes. The acetate moiety, perhaps in combination with existing hydroxyl groups, is an effective bond-forming substituent. The formation of strong molecular bonds leads to a very firm and intact tablet structure. Some fragmentation is induced by the slightly harder and more irregular shape of high-substituted starch acetate particles. The plastic flow under compression is enhanced. Acetylated material are slightly less sensitive to fast elastic recovery in-die, but somewhat more elastic out-of-die. In spite of their superior bonding, starch acetates under compression behave similarly to native starches. Drug Dev Ind Pharm. 2002; 28(2):165-75. Acetylation enhances the tabletting properties of starch. Raatikainen P, Korhonen O, Peltonen S, Parone P.

Agglomeration of powders containing starch acetate prior to tablet compression allows for modification and control of the release rate of the drugs from the starch acetate matrix tablets as well as the tensile strength of the tablets. J Pharm Sci. 2007 February; 96(2):438-47. Modifying drug release and tablet properties of starch acetate tablets by dry powder agglomeration. Mäki R, Suihko E, Rost S, Heiskanen M, Murtomaa M, Lehto VP, Ketolainen J.

Other Granule Excipients

Lactose is a milk sugar. It is a disaccharide composed of one galactose and one glucose molecule. In the pharmaceutical industry, lactose is used to help form tablets because it has excellent compressibility properties. It is also used to form a diluent powder for dry-powder inhalations. Lactose may be listed as lactose hydrous, lactose anhydrous, lactose monohydrate, or lactose spray-dried.

Various calcium phosphates are used as diluents in the pharmaceutical industry. Diluents are added to pharmaceutical tablets or capsules to make the product large enough for swallowing and handling, and more stable. Some calcium phosphate salts can be anhydrous, meaning the water has been removed from the salt form. Other calcium phosphates are termed dibasic, meaning they have two replaceable hydrogen atoms.

Hydroxypropyl cellulose (HPC) is a nonionic polymer, being a partially substituted poly (hydroxypropyl) ether of cellulose. It is available in different grades with differing solution viscosities. Molecular weight ranges from ˜80,000 to 1,150,000. High viscosity grades of HPC are generally used. Inclusion levels can vary from 15 to 40%. Addition of an anionic surfactant (e.g.,sodium lauryl sulfate) reportedly increases HPC viscosity and as a consequence reduces drug release rate. Combinations of HPC and other cellulosic polymers have been used to improve wet granulation and tableting characteristics and better control of drug release. HPC is thermoplastic and its presence may enable processing of HPMC containing formulations using hot melt extrusion or injection molding. It is not widely used because of its low swelling capacity and sensitivity to ionic strength of the dissolution media. Gel strengths of HPC matrices decrease during dissolution, leading to less cohesive gel structures. The lower tablet gel strength of HPC matrices, compared to HPMC can cause poor in vitro/in vivo correlation.

Hydroxyethyl cellulose (HEC) is also a nonionic, partially substituted poly (hydroxyethyl) ether of cellulose. It is available in several grades from Ashland Aqualon Functional Ingredients under the brand name of Natrosol®. These vary in viscosity and degree of substitution. High viscosity grades of HEC (1,500-5,500 mPa of 1% solution) are sometimes used in extended release formulations. Typical inclusion levels are 15-40% of the total formulation mass. However, it may be used in much lower levels such as the 2-3% used in the formulation of Example 1 of this invention. Swelling of HEC matrices has been reported to be considerably greater than HPC matrices. HEC matrices also exhibited relatively higher erosion rates, t50% (time to 50% release) being shorter for HEC than for HPC matrices.

Extra Granular Excipients

Gelling Agents

A carbomer is a homopolymer of acrylic acid, which is cross-linked, or bonded, with any of several polyalcohol allyl ethers. Carbomer is a generic name for synthetic high molecular weight polymers of acrylic acid. They may be homopolymers of acrylic acid, crosslinked with an allyl etherpentaerythritol, allyl ether of sucrose or allyl ether of propylene. In a water solution at neutral pH, carbomer is an anionic polymer. This makes carbomers polyelectrolytes, with the ability to absorb and retain water and swell to many times their original volume.

Poly acrylic acid and its derivatives are used in disposable diapers, ion exchange resins and adhesives. They are also popular as thickening, dispersing, suspending and emulsifying agents in pharmaceuticals, cosmetics and paints. Usually appearing as a white powder, the compound is used as a thickener and emulsion stabilizer. Best known for its use in the cosmetic industry, it also has practical applications in medicine and hygiene. wikipedia.com and wisegeek.com.

Similar to other polymers, carbomers are made of long chains of many smaller, repeating molecules, which have a large number of bonds. Although the molecular weight varies based on the exact molecules found in the chain, it typically is relatively high. These compounds are capable of absorbing large amounts of water, increasing in volume up to 1,000 times in some cases, so they can form gels and thick solutions that are stable and resistant to spoilage.

Scientists are able to make different types of carbomers, each of which has a slightly different molecular structure. To keep these different kinds straight, they use a numerical suffix and capitalize the word as in a proper title or name, such as Carbomer 940. Under this labeling system, the number indicates the average molecular weight of the polymer chains.

Carbomers are available from Lubrizol under the brand name of Carbopol® and are available in grades that vary in viscosity, polymer type, and polymerization solvent. Being cross-linked, these polymers are not water soluble but are swellable and gel forming. Swelling and gel formation behaviors differ somewhat from other hydrophilic polymers like HPMC, where swelling follows polymer hydration, leading to relaxation of polymer chains and their subsequent entanglement (physical crosslinking) to form a viscous gel. With acrylic acid polymers, surface gel formation is not due to polymer chain entanglement (the polymers are already cross-linked) but to formation of discrete micro gels comprising many polymer particles. Erosion, as occurs with linear polymers like HPMC does not occur because of the water insolubility. Instead, when the hydrogel is fully hydrated, osmotic pressure from within breaks up the structure, sloughing off discrete pieces of the hydrogel. The hydrogel remains intact and drug continues to diffuse uniformly through the gel layer.

In contrast to the situation with linear polymers, higher viscosity does not result in slower drug release with cross-linked polymers. Lightly cross-linked polymers (lower viscosity) are generally more efficient in controlling release than highly cross-linked variants. Release from carbomer matrices may depend on the pH of dissolution media, because of the anionic nature of the polymer (pKa 6±0.5). Swelling and gel formation are pH dependent. At lower pH the polymer is not fully swollen and drug release is faster. As pH increases the polymer swells and rapidly forms a gel layer, prolonging drug release. Carbomers, being anionic may form complexes with cationic drugs depending on drug properties such as pKa, solubility, amine group strength, steric orientation, molecular weight and size.

It has been reported that carbomer inclusion levels of about 30% produce comparable drug release profiles to HPMC in both water and 0.1 N HCl. Release was slower in pH 6.8 phosphate buffer. Carbomer matrices also exhibited significantly lower gel strengths compared to HPMC matrices in all three media. This has been postulated as the reason for their significantly faster drug release in vivo compared to HPMC matrices. 7 Drug-Polymer Matrices for Extended Release 143.

Polyethylene oxide (PEO) [POLYOX™] resins are water soluble, nonionic polymers manufactured by Dow Chemical Company . They are free flowing white powders, soluble in water at temperatures up to 98° C. and in certain organic solvents. Structures comprise the repeating sequence —(CH2CH2O)n where n represents the average number of oxyethylene groups. It is highly crystalline and available in molecular weight grades ranging from 1×105 to 7×106 Da. Their high molecular weights mean that the concentration of reactive end groups is very low. However, as their paired ether-oxygen electrons have a strong affinity for hydrogen bonding, they can form association complexes with a variety of monomeric and polymeric electron acceptors (e.g., gelatin, carbomer) as well as certain inorganic electrolytes, e.g., alkali halides. These water-soluble resins have applications in pharmaceutical products, such as in controlled release solid dose matrix systems, tablet binding, tablet coatings, transdermal drug delivery systems, and mucosal bioadhesives and gastro-retentive dosage forms. They exhibit film forming and water retention properties. It has high water solubility and low toxicity. http://www.pharmainfo.net/reviews/polyox-polyethylene-oxide-applications-pharma-industry. Submitted by Saritha R Bhandary on Wed, Sep. 22, 2010-22:38.

PEO resins are among the fastest hydrating water soluble polymers, quickly forming hydrogels that initiate and regulate drug release. Systems using such resins are often superior in approaching zero-order release profiles. PEO is generally used at 20-90% inclusion level depending on the drug and the desired release characteristics, however, in the instant invention levels of 10% and less are used.

PEO behaves similarly to HPMC in hydrophilic matrix systems. With appropriate selection of a suitable viscosity grade, one is able to achieve release profiles similar to hypromellose matrices. Grades available are POLYOX WSR-205 NF, WSR-1105 NF, WSR N-12 K NF, WSR N-60 K NF, WSR-301 NF, WSR-303 NF, and WSR Coagulant NF. The high swelling capacity of PEO has been used in hydrophilic matrices to achieve expanded swelling, providing enhanced gastroretention.

A formulation of gabapentin containing PEO and HPMC exhibited significant matrix swelling and gastric retention. 7 Drug-Polymer Matrices for Extended Release 145

Several other materials can be useful gel matrix formers. They include methylcellulose, guar gum, chitosan, and cross-linked high amylose starch.

Hydroxypropyl methylcellulose (HPMC or hypromellose) is a semisynthetic, inert, viscoelastic polymer used as an ophthalmic lubricant, as well as an excipient and controlled-delivery component in oral medicaments, found in a variety of commercial products. Hypromellose is a solid, and is a slightly off-white to beige powder in appearance and may be formed into granules. The compound forms colloids when dissolved in water. Wikipedia.com. It is widely used in matrix applications. Key advantages include global regulatory acceptance, stability, nonionic nature (resulting in pH-independent release of drugs), and ease of processing by direct compression (DC) or granulation. Other advantages are versatility and suitability for various drugs and release profiles (different viscosity grades being available) and extensive history of use. It is a mixed alkyl hydroxyalkyl cellulose ether containing methoxyl and hydroxypropyl groups. Type and distribution of the substituent groups affect physicochemical properties such as rate and extent of hydration, surface activity, biodegradation, and mechanical plasticity. Matrices exhibit pH-independent drug release profiles while aqueous solutions are stable over a wide pH range and are resistant to enzymatic degradation. Controlled Release in Oral Drug Delivery Clive G. Wilson Crowly Springer 2011 Chapter 7Drug-Polymer Matrices for Extended Release Sandip B. Tiwari, James DiNunzio, and Ali Rajabi-Siahboomi.

The first two digits represent the mean % methoxyl substitution and the last two the mean % hydroxypropyl substitution. HPMC is highly hydrophilic, hydrating rapidly in contact with water. Since the hydroxypropyl group is hydrophilic and the methoxyl group is hydrophobic, the ratio of hydroxypropyl to methoxyl content influences water mobility in a hydrated gel layer and therefore, drug release. Grades for extended release matrix formulations include E50LV, K100LV CR, K4M CR, K15M CR, K100M CR, E4M CR, and E1OM CR.Viscosities of 2% aqueous solutions of these polymers range from 50 to 100,000 cPs at 20° C. Inclusion level can vary from 10 to 80% dosage form.

Hypromellose in an aqueous solution, unlike methylcellulose, exhibits a thermal gelation property. That is, when the solution heats up to a critical temperature, the solution congeals into a non-flowable but semi-flexible mass. Typically, this critical (congealing) temperature is inversely related to both the solution concentration of HPMC and the concentration of the methoxy group within the HPMC molecule (which in turn depends on both the degree of substitution of the methoxy group and themolar substitution. That is, the higher the concentration of the methoxy group, the lower the critical temperature. The inflexibility/viscosity of the resulting mass, however, is directly related to the concentration of the methoxy group (the higher the concentration, the more viscous or less flexible the resulting mass is).

In addition to its use in ophthalmic liquids, hypromellose can be used as an excipient in oral tablet and capsule formulations, where, depending on the grade, it functions as controlled release agent to delay the release of a medicinal compound into the digestive tract. It is also used as a binder and as a component of tablet coatings.

Polymethacrylates are synthetic cationic or anionic polymers of dimethylaminoethyl methacrylates, methacrylic acid, and methacrylic acid esters in varying ratios. Methacrylic acid is a colourless, viscous organic acid with an acrid unpleasant odor. It is soluble in warm water and miscible with most organic solvents. Methacrylic acid is produced industrially on a large scale as a precursor to its esters, especially methyl methacrylate (MMA) and poly(methyl methacrylate) (PMMA). The methacrylates have numerous uses, most notably in the manufacture of polymers with trade names such as Lucite and Plexiglas. MAA occurs naturally in small amounts in the oil of Roman chamomile. Several types are commercially available (Eudragits®, Evonik) for use in drug formulations as dry powders and aqueous dosage forms. Polymethacrylates can be used as binders for both aqueous and organic solvent granulation, forming matrices with extended release characteristics. In general, greater polymer inclusion levels (5-20%) are used to control release from matrices. Drug release may also be affected by pH of the dissolution medium. Wiki.

Lubricants

Magnesium stearate is used as an anti-adherent and it has lubricating properties, preventing the ingredients from sticking to manufacturing equipment during the compression of powders into solid tablets. Magnesium stearate may also affect the release time of the cannabidiol in the tablets.

Coating

Opadry is A one-step film coating system which combines polymer, plasticizer and pigment. Opadry film coating resultS in attractive, elegant coatings that can be easily dispersed in aqueous or organic solvent solutions. Opadry results in the elimination of separate inventories of polymer, plasticizer and pigment and reduces batch-to-batch color inconsistency.

### EXAMPLES

The following examples illustrate various aspects of the present invention. They are not to be construed to limit the claims in any manner whatsoever.

### Example 1

In this example, immediate release cannabidiol in accordance with the present invention is prepared having the formula listed in Table 1:

TABLE 1

| Item | | |
|---|---|---|
| No. | Ingredient | Function |
| 1 | Cannabidiol extract | API |
| 2 | Dibasic Calcium Phosphate | Pharmaceutical |
| | Dihydrate (Emcompress ® Premium), | excipient Diluent |
| | NF | |
| 3 | Lactose Monohydrate (Granulac-200), | Pharmaceutical |
| | NF | excipient Diluent |
| 4 | Pregelatinized Modified Starch | Binder |
| | (Amprac-01), NF | |
| 5 | Hydroxy Ethyl Cellulose | Binder |
| | (Natrosol ® 250 HHX Pharm), NF | |
| 6 | Purified Water^{#} | |
| 7 | Carbomer Homopolymer, Type A | Release |
| | (Carbopol ® 71 G Polymer), NF | controlling agent |
| 8 | Polyethylene Oxide (Sentry (TM) | Release |
| | Polyox (TM) WSR Coagulant-Leo), | controlling agent |
| | NF | |
| 9 | Hypromellose (Benecel ® K200M | Release |
| | PH CR), NF | controlling agent |
| 10 | Methacrylic Acid Copolymer, Type C | Release |
| | (Eudragit ® L 100-55), NF | controlling agent |
| 11 | Magnesium Stearate, NF | Lubricant |
| TOTAL | | |
| 12 | Opadry II White (85F18422), INH | Film Coating |
| | | Material |
| 13 | Purified Water^{#}, USP | Coating |
| | | Solvent |

Drug formulations of the present invention are prepared as follows:

Granulating: the cannabidiol is blended with hydroxy ethyl cellulose, dicalcium phosphate, lactose monohydrate and pregelatinized starch acetate in a high shear granulator. The mixture is then granulated in hot water and dry mixed with the granulator and impeller set at slow speed. With the granulator and impeller set a slow speed, purified water is added to the mixed powders and granulated with the granulator and impeller set at slow speed.

Drying: The wet granulation is transferred to a fluid bed dryer and dried.

Mixing and tableting: After being dried in a fluid bed dryer, the granules thus formed are mixed with carbomer homopolymer, polyethylene oxide, hypromellose, methacrylic acid copolymer.

Coating: Finally, the tablets are coated with Opadry II White and other coating agents (e.g., talc and titanium dioxide) to produce the final formulation. The tablet granules were compressed into tablets on a tablet punching machine.

As will be clear to one skilled in the art having read the present specification, some of the steps may be carried out simultaneously or in a different order, such variations are included in the present invention.

All publications mentioned above are hereby specifically incorporated herein by reference in full for the teachings for which they are cited. The examples and claims of the present invention are not limiting. Having read the present disclosure, those skilled in the art will readily recognize that numerous modifications, substitutions and variations can be made to the description without substantially deviating from the invention described herein. Such modifications, substitutions and variations constitute part of the invention described herein.

What is claimed is:

1. A composition comprising a matrix tablet comprising granules of one or more cannabinoids mixed with starch acetate and extra granular excipients.

2. The drug composition of claim 1 wherein the cannabinoid is an extract from cannabis sativa.

3. The drug composition of claim 2 wherein the extract comprises more than 90% cannabidiol, THC or THCV.

4. The drug composition of claim 3 wherein the starch acetate comprises pregalatinized starch.

5. The drug composition of claim 4 wherein the granules further comprise diluents.

6. The drug composition of claim 5 wherein the diluents are selected from the group consisting of dibasic calcium phosphate and lactose.

7. The drug composition of claim 6 wherein the granules further comprise one or more additional non-ionic binders.

8. The drug composition of claim 7 wherein the additional non-ionic binder is hydroxyl ethyl cellulose.

9. The drug composition of claim 8 wherein the extragranular excipients comprise gelling agents.

10. The drug composition of claim 9 wherein the gelling agents are selected from the group consisting of carbomer homopolymers and polyethelene oxide.

11. The drug composition of claim 10 further comprising one or more additional release controlling agents.

12. The drug composition of claim 11 wherein the additional release controlling agents are selected from the group consisting of hypromellose and methacrylic acid copolymer.

13. The drug composition of claim 12 further comprising one or more lubricants and coatings.

14. The drug composition of claim 13 wherein the lubricant is magnesium sterate.

15. A drug composition comprising a monolithic matrix tablet comprising granules and extragranular excipients wherein the granules comprise:
a. cannabidiol or THC or THCV;
b. dibasic calcium phosphate;
c. lactose monohydrate;
d. pregelatinized modified starch; and
e. hydroxyl ethyl cellulose; and
wherein the extragranular excipients comprise:
a. carbomer homopolymer;
b. polyethylene oxide;
c. hypromellose; and
d. methacrylic acid copolymer.

16. A method for manufacture of a monolithic matrix drug composition comprising:
a. mixing a cannabidiol , dibasic calcium phosphate, lactose monohydrate, pregelatinized modified starch, and hydroxyl ethyl cellulose;
b. granulating the mixture with the solution to form a resulting mixture;
c. drying the resulting mixture and sizing the granules;
d. adding carbomer homopolymer, polyethylene oxide, hypromellose, and methacrylic acid copolymer to the granules; and
e. adding a lubricant to the dried mixture.
